(19) United States
(12) Patent Application Publication
(10) Pub. No.: US 2010/0137717 A1
(43) Pub. Date: Jun. 3, 2010

(54) Automatic Flow Tracking System and Method

(76) Inventor: Robert Strand, New South Wales (AU)

Correspondence Address: PATTERSON THUENTE CHRISTENSEN PEDERSEN, P.A., 4800 IDS CENTER, 80 SOUTH 8TH STREET, MINNEAPOLIS, MN, 55402-2100, US

(21) Appl. No.: 11/886,483

(22) PCT Filed: Mar. 15, 2006
(86) PCT No.: PCT/AU06/00338

§ 371 Date: Jul. 11, 2008

(30) Foreign Application Priority Data
Mar. 15, 2005 (AU) 2005901253

(51) Int. Cl. A61B 8/06; A61B 5/026; G06K 9/00

(52) U.S. Cl. 600/454; 600/508; 382/128

(57) ABSTRACT

A method of determining the blood flow characteristics from a monitoring signal indicative of blood flow in the vicinity of a heart, the method including the steps of: (a) extracting a flow envelope from the monitoring signal; (b) extracting a series of temporal markers from the flow envelope. (c) removing extraneous flows such as valve opening and closing flows from the flow envelope; (d) extracting features from the flow envelope and monitoring signal, such as peak velocity; (e) Producing cardiac parameters based on said monitoring signal.

### RELATED APPLICATIONS

This application claims priority to PCT Application No._PCT/AU2006/000338 filed Mar. 15, 2006, and Australian Application No. 2005901253 filed Mar. 15, 2005, the disclosures of which are hereby incorporated by reference in their entireties.

### FIELD OF THE INVENTION

The present invention relates to the field of Doppler flow measurements of blood flow and, in particular, to the automatic tracking of the envelope of a Doppler flow spectral profile for extracting relevant cardiac parameters in real-time. The method can also be applied to non-real time analysis.

### BACKGROUND OF THE INVENTION

Image recognition and processing techniques are increasingly becoming important in many aspects of clinical diagnosis and monitoring. In cardiography, non-invasive methods such as Doppler flow echocardiography are gaining preference over invasive techniques, particularly for seriously ill patients. However, the extraction of relevant cardiac measures from the irregular profile of the Doppler flow spectrogram still relies on the manual outlining of the flow regions which is time-consuming and labour intensive. As such, this method is not suitable in its present form for continual real-time monitoring of the cardiac health of a patient.

Automatic trace algorithms of Doppler spectrograms are known; however, they consistently either over- or under-estimate the actual flow contained in the system. This is mainly due to flows and artefacts inherent in the heart, particularly at the instants of valve opening and closing, and the presence of noise in the recorded signal. Thus, the results are of limited use.

Techniques for Doppler ultrasound cardiography and determination of maximum frequency via spectral estimation methods are known. There are, for example, the systems described in: “A New Approach for Determining Maximum Frequency in Clinical Doppler Ultrasound Spectral Estimates” Aaron H. Steinman, Jahangir Tavakkoli, Jerry G. Myers Jr., Richard S. C. Cobbold, C, K. Wayne Johnston, 2000, Journal Unknown [Ref. 1]; “Comparison of four digital maximum frequency estimators for Doppler ultrasound”, L. Y. L. Mo, L. C. M. Yun, and R. S. C. Cobbold, Ultrasound Med. Biol. 1988, 14(5), p. 355-363 [Ref. 2]; “Comparison of the performance of three maximum Doppler frequency estimators coupled with different spectral estimation methods”, K. Marasek and A. Nowicki. Ultrasound Med. Biol. 1994, 20(7), p. 629-638 [Ref. 3]; “Continuous Display of Peak and Mean Blood Flow Velocities”, U.S. Pat. No. 5,287,735, 1994, Helen F. Routh, Charles W. Powerie Jr, Roy B. Peterson [Ref. 4]; “Development of a Method For Calculation of Cardiac Output Using Doppler Ultrasound”, Amit Diggikar, B. E. WEST VIRGINIA UNIVERSITY, Master of Science in Electrical Engineering Thesis, 1999 [Ref. 5]; and “Beat-to-beat detection of fetal heart rate: Doppler ultrasound cardiotocography compared to direct ECG cardiotocography in time and frequency domain.”, Chris H L Peters, Edith D M ten Broeke, Peter Andriessen, Barbara Vermeulen, Ralph C M Berendsen, Pieter F FWijn and S Guid Oei, Physiol. Meas. (2004) 585-593 [Ref. 6] It is desirable that a system for extracting the relevant cardiac parameters can be reliably extracted from the Doppler flow profile to enable real-time non-invasive monitoring of a patient and rapid detection and response to any abnormalities detected on a beat-to-beat basis.

### SUMMARY OF THE INVENTION

It is an objective of the present invention to provide a method of automatically tracking a fluid-flow profile and particularly for tracking a Doppler signal from a fluid flow in a heart and extracting relevant cardiac monitoring parameters.

In accordance with a first aspect of the present invention, there is provided a method of determining the blood flow characteristics from a monitoring signal indicative of blood flow in the vicinity of a heart, the method including the steps of: (a) extracting a flow envelope from the monitoring signal; (b) extracting a series of temporal markers from the flow envelope; (c) removing extraneous flows such as valve opening and closing flows from the flow envelope; (d) extracting features from the flow envelope and monitoring signal, such as peak velocity.

The method can also include the step of smoothing the flow envelope. The monitoring signal can comprise an ultrasound signal indicative of blood flow in the vicinity of a heart and the method provides for the real time monitoring of blood flow characteristics of a patient's heart. The cardiac monitoring parameters are calculated based on information extracted in steps (a), (b), (c) and (d) which then may be used to calculate further cardiac related parameters. The cardiac monitoring parameters are preferably extracted in real time.

The maximum flow rate can be derived substantially from a maximum frequency signal level present. For rectangular cross-sectional velocity profiles the maximum flow rate corresponds to the blood flow in the cardiac system. To those skilled in the art it is obvious that other methods can be used to determine blood flow which are more suitable to different underlying cross-sectional flow profiles.

The step (a) further preferably can include the step of extracting the flow rate of fluid within the heart as a function of time. The step (a) preferably can include the steps of: (a1) forming a frequency domain transform of the monitoring signal; (a2) examining the frequency domain characteristics of the monitoring signal to determine a flow rate. The step (a2) preferably can include the step of adjusting the determination in accordance with signal to noise levels in the monitoring signal.

The step (a2) can preferably include steps of (a2.1) determining the power spectra and integrated power spectra from the FFT magnitudes; (a2.2) estimating the noise power in the power spectra; (a2.3) determining the slope threshold for the integrated power spectra use to find the actual flow represented by the Doppler signal. Further including bias correction factors to scale an inaccurate quantity to a correct value without increasing the sensitivity to noise.

In accordance with a further aspect of the present invention, there is provided an automatic cardiac monitoring device including: a. means of detecting a Doppler spectral signal; b. means for processing the Doppler spectral signal to automatically extract a plurality of cardiac parameters; c. means for identifying timing markers in the processed Doppler signal to extract required cardiac parameters; d. means for simultaneously displaying a visual representation of the processed Doppler signal and the timing markers.

The processing means preferably can include: a method for extracting a flow envelope from the Doppler signal; and a transfer function for removing undesired flow signals and artefacts from the flow envelope. The cardiac parameters are preferably extracted in real-time.

The device can be computationally compatible with fixed point DSP processors.

The advantages and characteristics of the present invention will be illustrated and described, but not limited, in the following preferred embodiments, with reference to the accompanying drawings.

### BRIEF DESCRIPTION OF THE FIGURES

Preferred embodiments of the present invention will now be described with reference to the accompanying drawings, wherein:

FIG. 1 is a Doppler signal processed in accordance with the present system and showing relevant extracted cardiac parameters;

FIG. 2 is a simplified graph of a typical power spectrum, noise spectrum and power spectrum, obtained from a Doppler spectrograph;

FIG. 3 is a graph of the integrated power spectrum (IPS) from the graphs of FIG. 2;

FIG. 4 is a graph of a power spectra obtained from the present system of near the peak of the flow;

FIG. 5 is an enlarged view of the graph of FIG. 4;

FIG. 6 is a typical Doppler flow signal;

FIG. 7 is the Doppler flow signal of FIG. 6, processed with a rectangular FFT window with N=16;

FIG. 8 is the Doppler flow signal of FIG. 6, processed with a Kaiser FFT window with N=20 and β4;

FIG. 9 is a graph of the typical noise level of the present system;

FIG. 10 is a graph of the Doppler flow signal of FIG. 6 and an initial trace of the flow envelope to remove signals from the unwanted low velocity regions of the flow profile;

FIG. 11 is a region of the Doppler flow signal of FIG. 6 showing a single heart beat and identifying the signals due to valve-opening and closing, the underlying desired flow profile and the relevant timing markers;

FIG. 12 is a table of Samples vs. Heart Rate for the system of the preferred embodiment;

FIG. 13 is a graph of the Doppler flow signal of FIG. 6 showing the approximate markers used to extract the cardiac parameters;

FIG. 14 is the region of FIG. 11 showing the improved indication of valve activity from a series of thresholds: (a) Total Power no threshold, (b) Valve Power (threshold=128), (c) Valve Power (threshold=192);

FIG. 15 is a graph of the Doppler flow envelope showing the valve closure markers;

FIG. 16 is a table of extracted cardiac parameters for the Doppler envelope of FIG. 15;

FIG. 17 is a graph of the Doppler flow profile of FIG. 15 with Gaussian noise added (noise=4);

FIG. 18 is a graph of the Doppler flow profile of FIG. 15 with uniform noise added (noise=7);

FIG. 19 is a graph of the Doppler flow profile of FIG. 15 with Gaussian noise added (noise=2.3);

FIG. 20 is a table of SNR characteristics showing the effect of adding noise the Doppler Signal;

FIG. 21 is a table of extracted cardiac parameters for the Doppler profile of FIG. 17;

FIG. 22 is a table of extracted cardiac parameters for the Doppler profile of FIG. 18;

FIG. 23 is a table of extracted cardiac parameters for the Doppler profile of FIG. 19;

FIG. 24 is a table of Low-Noise to Added-Noise quantity ratios with Gaussian noise added (noise=4);

FIG. 25 is a table of Low-Noise to Added-Noise quantity ratios with uniform noise added (noise=7);

FIG. 26 is a table of Low-Noise to Added-Noise quantity ratios with Gaussian noise added (noise=2.3);

FIG. 27 is a perspective view of a patient utilising the device of the preferred embodiment;

FIG. 28 is an illustration of an example doppler output;

FIG. 29 is a schematic view of the hardware functionality of one, form of the preferred embodiment; and

FIG. 30 is an illustration of the steps of the preferred embodiment.

### DESCRIPTION OF PREFERRED AND OTHER EMBODIMENTS

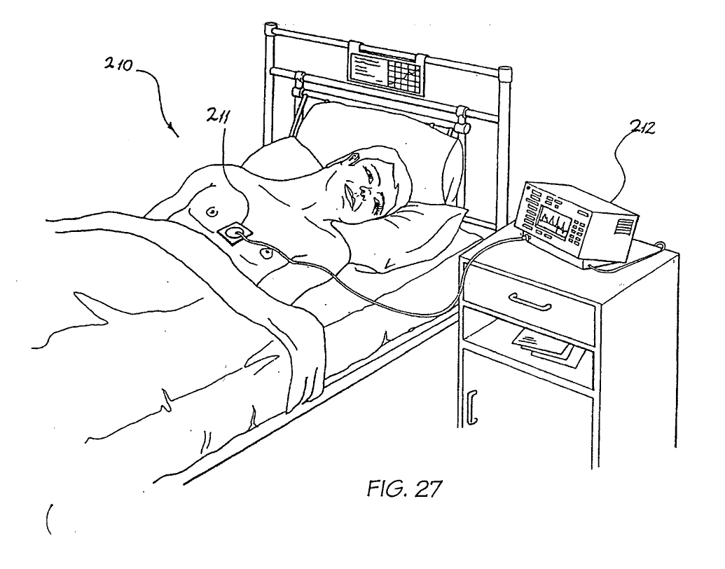

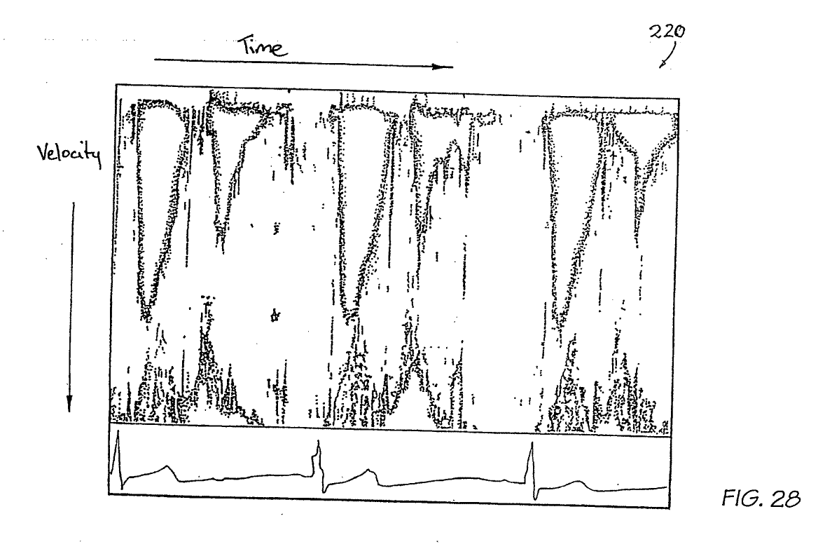

The preferred embodiment is designed to operate with a suitably modified cardiac monitoring systems such as those disclosed in U.S. Pat. No. 6,565,513 entitled “Ultrasonic Cardiac Output Monitor”, the contents of which are hereby incorporated by reference in their entirety. FIG. 27 illustrates the operation of a system utilised in accordance with the aforementioned patent application. The cardiac output of the heart of a patient 210 is monitored utilising an ultrasonic transducer 211 attached to a processing device 212 which processes the return signal from the transducer to provide an output trace 220, illustrated in FIG. 28.

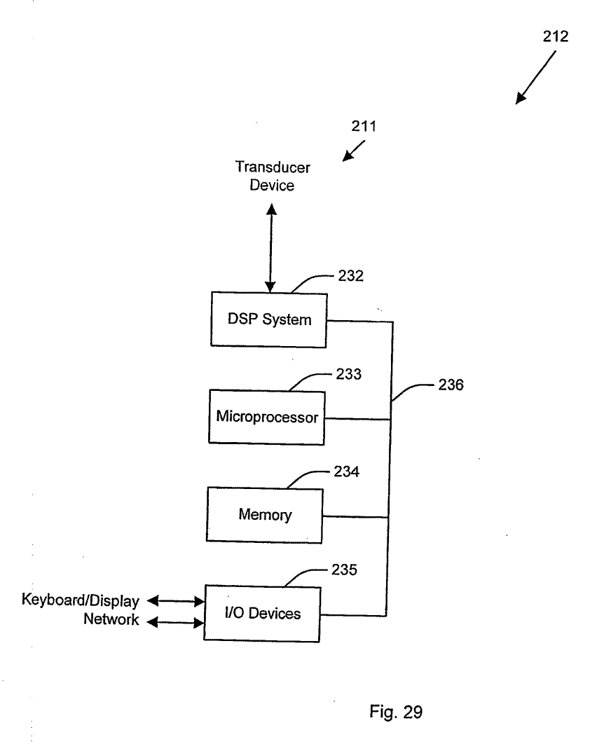

The processing device 212 can be structured as illustrated schematically in FIG. 29 with the transducer 211 interconnected to a Digital Signal Processing device 232. The DSP device is further interconnected to a microprocessor 233, memory 234 and I/O device controller 235 by means of bus 236. It will be obvious to those skilled in the art that other architectures would be possible.

The purpose of the automatic flow tracking system of the preferred embodiment is to trace the signal received from a Doppler flow profile and to extract cardiac parameters from the Doppler spectra. The trace algorithm also provides a visual trace of the Doppler flow profile but it is the cardiac parameters extracted from the tracing which provide the useful quantitative outputs.

The preferred embodiment is constructed from suitable programming of the microcontroller of the above mentioned hardware arrangement.

### SUMMARY OF TERMINOLOGY

The following terms are utilised throughout the specification:

ET Ejection Time

FFT Fast Fourier Transform

HR Heart Rate

IPS Integrated Power Spectrum

Pₘₙ Mean Pressure Gradient ( )

MTCM Modified Threshold Crossing Method

Vₚₑₐₖ Peak Velocity

PS Power Spectrum

SNR Signal to Noise Ratio

SNSI Signal Noise Slope Intersection Method

Spoke A set of spectral estimates at a point in time

VTI Velocity Time Integral

The automatic flow tracking system extracts the temporal and flow-derived cardiac parameters from a Doppler spectral display. This is done using an algorithm to trace the recorded flow profile, from which the desired cardiac parameters are calculated. The cardiac parameters are then used by a main application to calculate further cardiac related parameters.

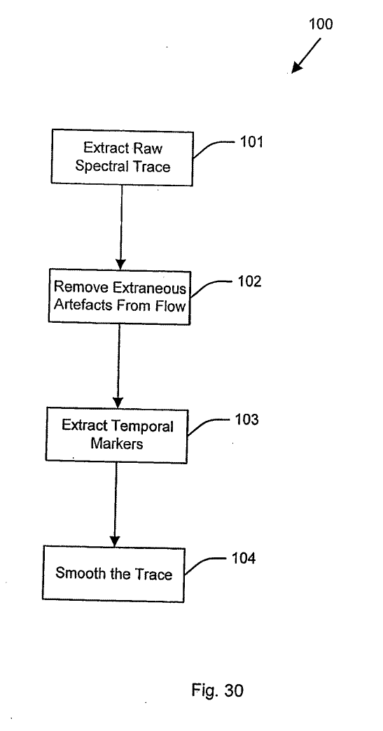

In the preferred embodiment, it is assumed that the Doppler return signal is the only input to the system and all parameters and features are extracted from this signal. FIG. 30 illustrates the steps involved in the preferred embodiment. These include the steps of extracting a raw spectral trace 101, extracting temporal markers 103, removing extraneous artefacts from the flow 102 and smoothing the trace 104.

Raw Spectral Trace 101: Extraction of the flow envelope from the Doppler spectral data at each point in time. No attempt is made to remove extraneous flows and artefacts from the trace.

Temporal Marker Extraction 103: Time markers used to estimate Heart Rate and Ejection Time. This phase is integrated into the Valve Removal phase since it uses common timing markers.

Valve and Non-flow Removal 102: This stage of the algorithm removes the extraneous flows and artefacts from the raw spectral trace leaving only the wanted flow.

Trace Smoothing 104: The trace is smoothed to provide a visually pleasing trace.

Each of these stages is discussed in further detail hereinafter.

Initially, the Doppler signal is converted to a spectrogram using the FFT. The relationship between blood velocity and frequency is determined from the standard Doppler equation.

For a continuous wave Doppler system the spectrum at each point in time contains a spread of frequencies. From research studies on Transvalvular CW Doppler the maximum frequency, at each point in time, in the spread of frequencies is a good estimate for the true blood velocity when the crossectional flow profile is rectangular.

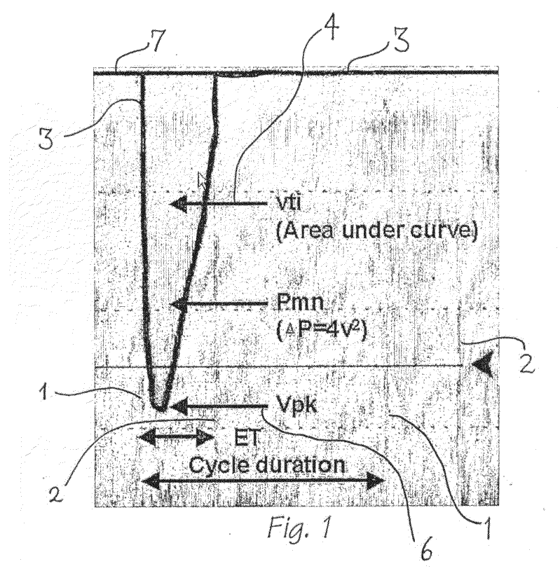

FIG. 1 shows an example close up view of the Doppler spectrum for the pulmonary valve taken from the parasternal position. A number of cardiac parameters may be extracted from the features of the Doppler spectrum.

Temporal Cardiac Parameters:

Heart Rate (HR): Rate at which the heart pumps blood.

$$H R = \frac{60}{CycleTime}$$

where, HR is given in beats per minute [bpm] and Cycle Time is given in seconds.

Ejection Time (ET): The period of time blood is ejected through the output tract.

The main interest is to measure blood flow through targeted vessels or valves. The Doppler spectrum also contains Doppler-shifted signals from a number of other extraneous sources. In order to determine blood contributing only to wanted flow, the effect of these extraneous sources must be removed. Extraneous sources include:

Returns from moving tissue, shown as low-frequency spectra. To a large extent these have been removed from FIG. 1 by a wall filter-which is evident from absent low frequency band.

Returns from blood flow between the atria and the ventricles.

Returns from moving Valve leaflets. These can be seen as spikes 1 and 2, respectively on the leading edge (valve-opening) and trailing edge (valve-closure) of the flow profile.

The trace 3 on FIG. 1 shows only the wanted flow through the pulmonary valve. Notice the trace follows only the flow between the valve-open and valve-closure regions. The extraneous flows and valve clicks have been removed. Once a trace of the wanted flow is found a number of flow-derived Cardiac parameters can be calculated. For the cardiac parameters to be meaningful, the velocity flow profile v(t) is the underlying flow when the extraneous sources have been removed.

Flow-Derived Cardiac Parameters:

Velocity Time Integral 4 (VTI): Represents the distance the blood flows during a single cardiac cycle.

$$V T I = {\int }_{0}^{ET} v ( t ) d t$$

Mean Pressure Gradient 5 (Pₘₙ): Represents the mean pressure difference across the valve during ejection.

$${P}_{mn} = Δ P = 4 \overline{{v ( t )}^{2}} = \frac{4}{E T} {\int }_{0}^{ET} {v ( t )}^{2} d t$$

Note: the factor of 4 above combines blood density and conversion to mmHg.

Peak Velocity 6 (Vₚₖ): Peak flow velocity over the ejection period.

$${V}_{pk} = {max}_{l ∈ [ 0 , ET ]} ( v ( t ) )$$

In order to calculate the VTI, which is required to calculate the cardiac output, the velocity profile v(t) must be estimated. The quantity v(t) must be extracted from the frequency peaks of the spectrogram using a maximum frequency detector.

Established algorithms exist for maximum frequency detectors which are specifically aimed at ultrasound spectrograms. The accuracy of the algorithms depend largely on the signal to noise. None of these algorithms are deemed perfect solutions and it is inevitable new algorithms will appear in the future. The basic algorithms do not remove flow artefacts.

The automatic flow tracking system of the preferred embodiment is designed to be operated with the following constraints:

Real Time operation.

Beat-to-Beat parameter extraction of cardiac function.

Minimal delay from Real Time events to feature identification.

Computationally compatible with existing Fixed Point DSP Processor systems.

Raw Spectral Trace

### BACKGROUND

In the following it is assumed that the quantities and FFT bins correspond to a selected direction of flow. Traditionally the power spectrum is integrated to form the integrated power spectrum (IPS),

$$I P S ( k ) = {\sum }_{j = {k}_{l}}^{k} P ( j )$$

where, kₗ is the FFT bin index corresponding to the wall-filter cut-off (if desired), P(k) is the FFT bin power. As defined, IPS(k) includes the contribution of P(k).

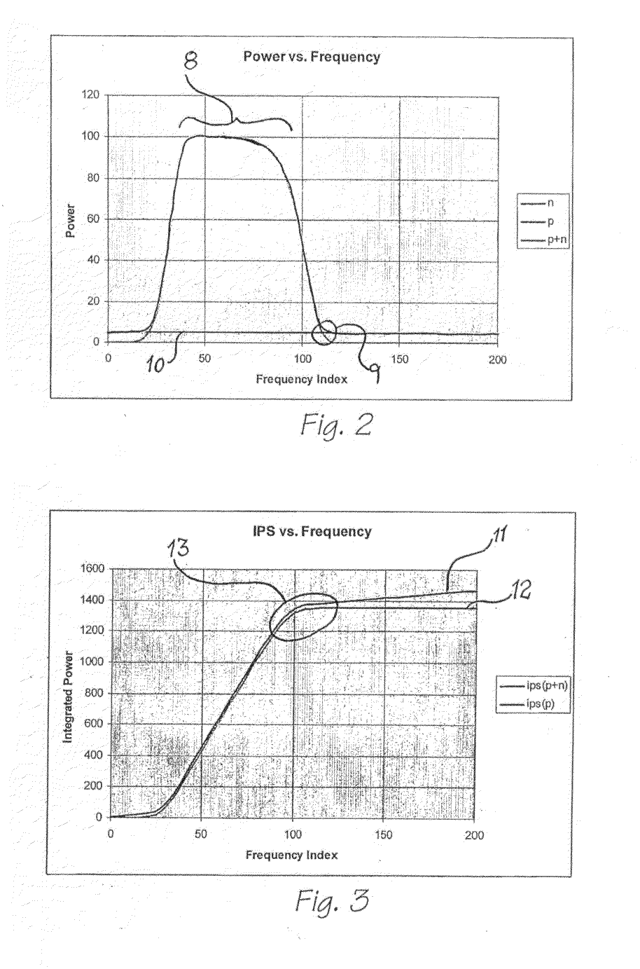

FIG. 3 shows the IPS corresponding to FIG. 2 for both the signal alone (11) and the signal and noise together (12). The local slope of the IPS represents the power level at a particular frequency. At higher frequencies, which are above the maximum flow, the slope of the IPS approaches the signal's noise power. The discrete slope can be defined as,

m(k)=IPS(k)−IPS(k−1)

This definition ensures the k^{th} slope equals the k^{th} discrete power value ie. m(k)=P(k).

The majority of maximum frequency detection algorithms decide the maximum frequency by finding the knee in the IPS (13 of FIG. 3). The difference between the algorithms is the way this is done. Whilst the techniques are outlined in the aforementioned references, a discussion follows of the technique used in a first embodiment.

Algorithm

There are a number of problems involved with the tracing of the Doppler flow profile when using the variety of known methods with the present device. These include:

Definition of a suitable slope threshold.

Spectral smoothing.

The estimation of the signal levels.

The estimation of noise levels.

Miscellaneous issues.

These issues and their respective solutions are discussed below.

### Basic Definitions

The input to the algorithm is the FFT magnitudes fft(k). The trace algorithm only operates on one side of the spectra at a time. Algorithm parameters include:

fₕ Upper frequency bound for spectra and IPS analysis; bin index units.

fₗ Lower frequency bound for spectra and IPS analysis (typically this is set to 0, but can also be used to keep prevent the search traversing the wall filter region); bin index units.

f_{Nl} Start of flat noise region above the 1/f region; bin index units.

Nₛₚₐₙ Number of points to perform rectangular filtering of power spectra (typically 17).

N_{sig} Span of points used to compute local signal level (typically 25).

k_{N} Noise factor for slope threshold.

kₛ Signal factor for slope threshold.

The Power Spectra (P) can be computed from the FFT as follows:

$$P ( k ) = \{ \begin{matrix}{fft ( k )}^{2} ; & if fft ( k ) \leq {fft}_{max} \\ {fft}_{max}^{2} ; & otherwise\end{matrix} \\ k = 0 to {f}_{h} - 1$$

where fftₘₐₓ is an algorithm parameter which clamps the power spectra values (typically 32 to 255).

The Integrated Power Spectra (IPS) is:

$$I P S ( k ) = {\sum }_{j = {f}_{l}}^{k} P ( j )$$

$$k = {f}_{l} to {f}_{h} - 1$$

Slope Threshold

A generalized form of slope threshold was used:

mₜₕᵣₑₛₕ=kₛmₛ+k_{N}m_{N}

where kₛ is a signal weighting factor and k_{N} is a noise weighting factor, m_{N} is the noise power estimate and mₛ is the signal power estimate. This threshold depends on both noise and signal strength but has independent control over the weighting factors. The factor k_{N} controls the susceptibility to false detection in the noise region.

Spectral Smoothing

The captured raw spectra from the FFT can be very rough, containing many peaks and troughs across the spectra. The signal to noise on Doppler systems can be low which requires the detection thresholds to be close to the noise floor. With the rough spectra, large peaks in the noise region can exceed the low detection threshold and cause the maximum frequency detector to misdetect the edge of the spectra.

There are a number of established methods to reduce the variance in power spectrum estimates. Most of these methods require additional storage and computation. One method of smoothing is to take multiple FFT's over time and average the results.

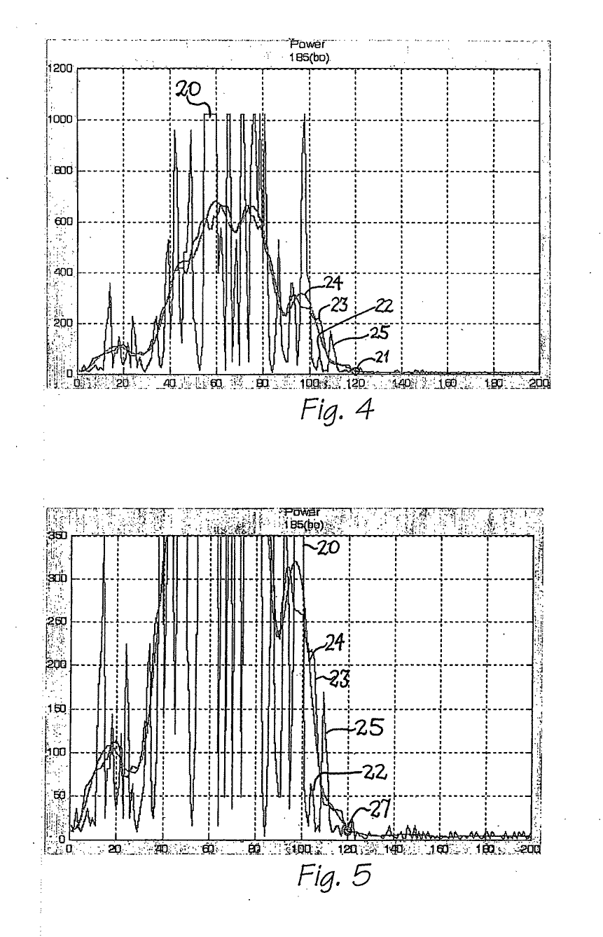

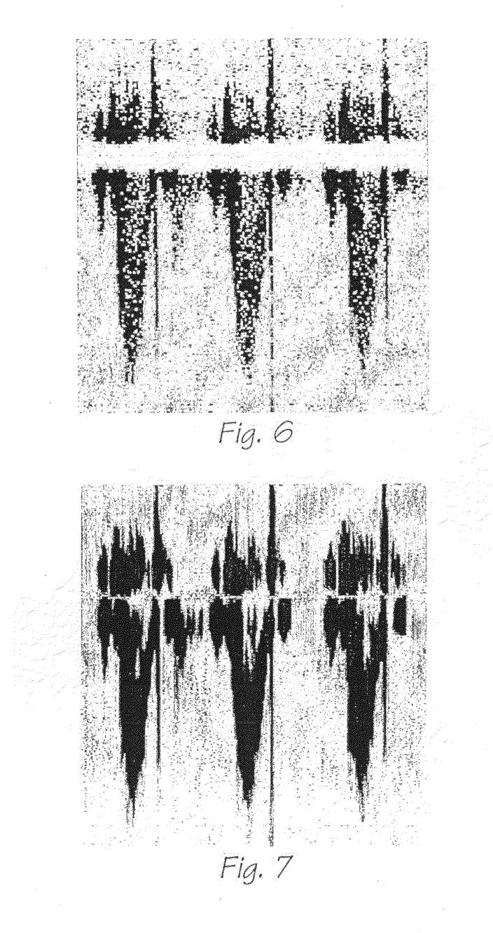

An alternate is to smooth the power spectra in frequency. The smoothing can be done using a sliding window function which essentially provides a local average of the spectra across a few bins. FIG. 4 and FIG. 5 show a smoothed power spectra: curve 23 is for a rectangular window of length 16, and curve 24 is for a Kaiser window of length 20 and β=4 (is a parameter for the Kaiser window). The two filters have roughly the same of the main lobe width (and hence effectively average over a similar number of points).

Applying the windowing/filtering to the power spectra requires a significant amount of processing because the smoothing filter must be applied to the complete set of spectral values.

One way of minimizing computations is to calculate the IPS slope from IPS values spanning a number of bins. This turns out to be equivalent to smoothing the power spectra with a rectangular filter then computing the single point IPS slope over a span of one bin.

Consider the case of the unsmoothed spectra and spanned IPS slope calculation. The slope of the line between points k and k+n on the IPS is a central difference estimate for the slope at the mid-point of k+1 and k+n,

$$m ( k + \frac{n + 1}{2} ) = \frac{I P S ( k + n ) - I P S ( k )}{n}$$

Shifting this down to get the slope at k,

$$\begin{matrix}m ( k ) = \frac{I P S ( k + n - \frac{n + 1}{2} ) - I P S ( k - \frac{n + 1}{2} )}{n} \\ = \frac{I P S ( k + \frac{n - 1}{2} ) - I P S ( k - \frac{n - 1}{2} - 1 )}{n} \\ = \frac{1}{n} ( {\sum }_{j = 1}^{k + \frac{n - 1}{2}} P ( j ) - {\sum }_{j = 1}^{k - \frac{n - 1}{2} - 1} P ( j ) ) \\ = \frac{1}{n} {\sum }_{j = k - \frac{n - 1}{2}}^{k + \frac{n - 1}{2}} P ( j )\end{matrix}$$

Now consider the case where the spectra is smoothed first. The power spectra smoothed over a symmetric (2N+1) point rectangular window is,

$${P}^{′} ( k ) = \frac{1}{2 N + 1} {\sum }_{j = k - N}^{k + N} P ( j )$$

The corresponding IPS is,

$$I P {S}^{′} ( k ) = {\sum }_{i = 0}^{k} {P}^{′} ( i )$$

Hence the slope over one bin span is,

m′(k)=IPS′(k)−IPS′(k−1)=P′(k)

If we choose n=2N+1 then,

$${P}^{′} ( k ) = \frac{1}{n} {\sum }_{j = k - \frac{n - 1}{2}}^{k + \frac{n - 1}{2}} P ( j )$$

Hence, when n is odd, the two slopes m(k) and m′(k) are identical.

A slight improvement on the rectangular filter is the modified Daniell window. The coefficients for the modified Daniell window are the same as the rectangular window except the two end point weights are half that of the centre points. This window can be implemented by subtracting half the sum of the two power spectra end-points from the efficiently computed rectangular window. The window scaling factor can be ignored provided all values are modified Daniell window filtered.

The side effect of spectral smoothing is that sharp transition edges are smeared. For an ideal transition, the rectangular filter skews chosen points when the threshold is not 50% of the size of the transition edge.

Signal Levels

The way the signal level is estimated can change the way the algorithm behaves.

For example, the SNSI method estimates the signal level around the peak power. Occasional very strong signals can cause the signal dependent threshold to rise which in turn causes the detected maximum frequency to be too low. This shows-up as a “sucked out” region in the trace envelope.

One means of limiting the magnitude of the signal is to clamp the levels. The general flow levels are much lower than the peaks and clamping provides a better estimate of flow signal strength.

Many cardiac related targets do not produce a well defined spectral peak just below the spectral edge; this is particularly true for CW Doppler. Quite often there are strong low frequency components and gradually diminishing signal power as we approach the edge. The strong low frequency components do not represent the signal level well. The average signal level just below the edge appears to be a more useful estimate of the signal level.

The averaging effect of the filter used to smooth the power spectra helps with the fuzzy tips case. The weak signal, which is over the top of the noise speckle, is converted to a smooth transition from signal to noise.

Noise Estimate

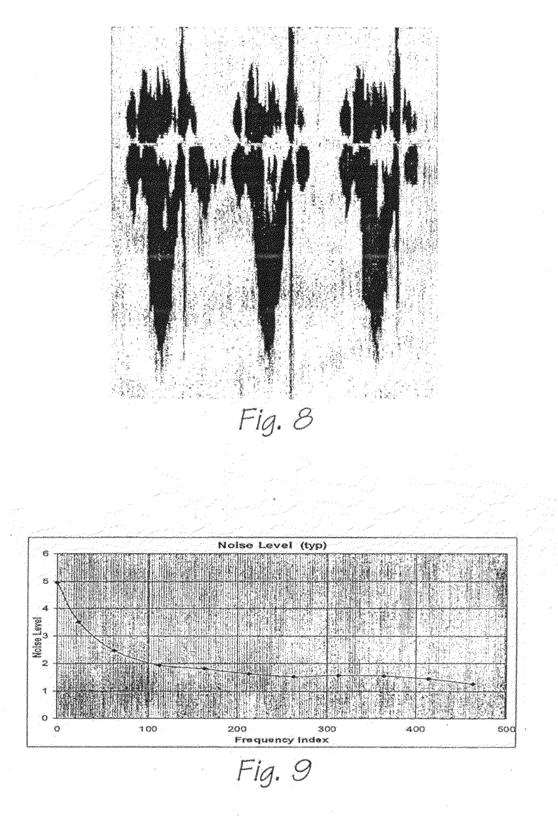

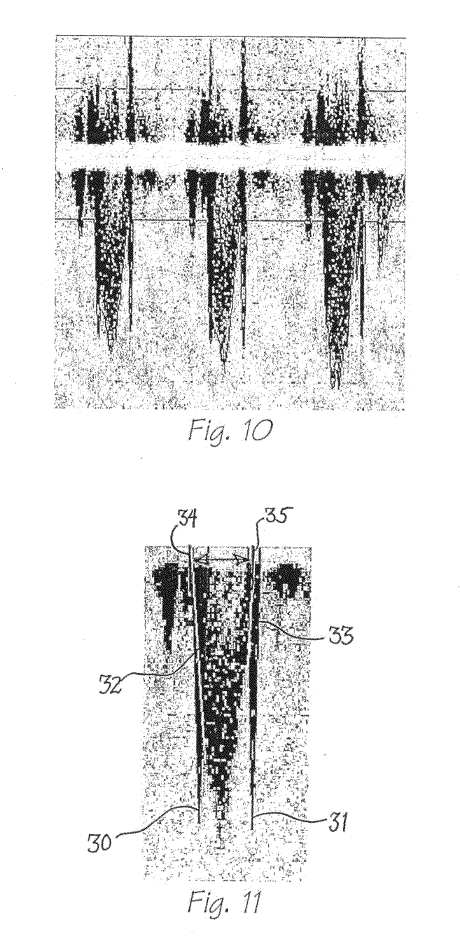

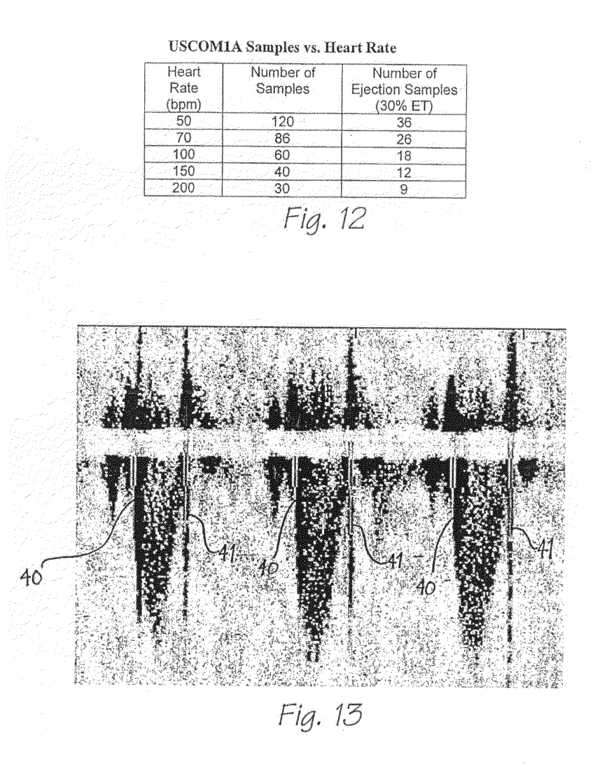

The noise spectrum from the utilised device was found not to be constant with frequency, instead, the noise followed a “1/f”-type characteristic. The noise was found not to precisely follow a 1/f curve but has the general characteristic of increased noise as frequency drops below some particular frequency, and above that frequency, the noise flattens off to a near constant level. FIG. 9 shows the typical noise level of the device recorded with a transducer.

The noise power reference is estimated from the relatively flat tail of the power spectra. The noise power is the slope of the IPS from the start of the noise region f_{Nl} to the end of the noise region is fₕ.

The start of the noise region is taken as the start of the 1/f noise region f_{Nl}. If there is high velocity flow present the signal peaks may exceed f_{N1}, and the noise estimate will become contaminated with signal. To prevent this, the start of the noise region is set to a point above the maximum frequency. Because the maximum frequency is not known, an estimate must first be made for start of the noise region. The following scheme was found to work:

The smoothed power spectra is searched for the maximum power, the corresponding frequency index is f_{ps max}. The IPS is smoothed using the smoothing window.

If maximum signal power is greater than the last noise estimate by the factor k_{Nu}, then there is enough signal to determine a boundary, otherwise the signal level is below the noise and there is no discernable boundary:

f_{Nl}′=f_{ps max}+(fₕ+f_{ps max})/3

where f_{ps max} is the position of the maximum power point, and after which apply a clamp,

f_{Nl}′←f_{Nl}; f_{Nl}′≦f_{Nl}

If the signal level test failed, use the top of the spectra f_{Nl},

f_{Nl}′=f_{Nl}

Once the lower frequency limit of the noise floor f_{Nl}′ is determined, the noise power m_{N} is estimated by averaging the bins from f_{Nl}′ to fₕ. To ensure accuracy and to avoid fractional values the noise estimate, the noise power is represented as a sum of bins f_{Nl}′ to fₕ. The sum is then scaled to represent the average over Nₛₚₐₙ bins.

If the noise estimate is more than twice the previous (spoke) noise estimate the noise is limited to twice the previous (spoke) noise estimate. This prevents small scratches and anomalies that extend into the noise region affecting the noise estimate.

To further reduce the variance of the noise estimate, the noise is averaged over a number of spokes ie. over time. A moving average is used. The larger the number of spokes the lower the variance. However, the noise is not stationary, typically the noise was found to fluctuate due to transducer coupling and transducer movement. If the averaging period is too long the spoke noise could be much higher than the averaged noise which will cause a false detection in the noise region. The number of spokes used to average the noise was set to 4.

The adjusted noise estimate account for the 1/f type variation of noise with frequency. The idea is the tail of the region is flat with frequency, and is mostly unaffected by signals, which allows the tail region to be used as a measure of the noise level. The noise profile is largely due to the processing chain hence the noise can be modelled. The noise tail can be used to adjust the overall level.

The noise power reference is scaled using a multiplicative term,

M_{N}′=k_{Nf}m_{N}

where m_{N} is the noise estimated in the noise tail, and k_{Nf} is a function of the frequency index i. k_{Nf} may be represented in tabular or functional form. One form is a piecewise linear approximation,

$${k}_{Nf} = \{ \begin{matrix}1 ; & for i > 250 \\ ( 2 + 100 / 150 - i / 150 ) ; & for i <= 250 and i > 100 \\ ( 3 + 50 / 50 - i / 50 ) ; & for i <= 100 and i > 50 \\ ( 6 - 3 * i / 50 ) ; & for i <= 50 \\ 200 ; & for i < fl\end{matrix} \\ Note \text{:} For i < {f}_{l} , a better value is ( 8 * 50 - 5 * fl ) ) / 50.$$

Rejection of False Detections

Doppler systems process very low level signals which make them prone to interference. Interference is often seen as horizontal lines where the lines correspond to strong interference spectra. The lines may vary in spectral width and can also wander in frequency. The spectral lines can affect the trace algorithm. A spectral smoothing helps average out thin noise lines but is less effective on wider spectral lines.

False detection is done using two checks. The first check is a rough check to prevent false detection due to inadequate noise and signal. At each frequency the “general” signal to noise around the current point is checked to be greater than a certain value. The signal level m_{S} is calculated using a wider span of bins than m_{N}′ but the value is scaled back to the same number of bins.

The second check is to prevent false trips due to strong interference lines. The slope threshold is recalculated based on the signal somewhat below the current edge point. A slightly higher noise weighting constant is used for the threshold based on the assumption that the signal levels increase at lower frequencies. The signal calculated this way should be below the region of a noise line. The noise level used is the adjusted noise based around the edge point. The check is not done when the signal estimate position is below the system wall filter frequency.

Edge Refinement

As discussed earlier the spectral smoothing smears the edge of the transition. This is particularly evident on well defined edges with high signal to noise. An edge refinement stage attempts to locate the edge in the vicinity of where the slope threshold was exceeded. The edge refinement step is based on the centroid of signal power over the smoothing span. This method has a bias which needs to be corrected. When the signal is strong and the slope threshold is low, the bias is minimal.

Bias Correction Factors

The detected raw trace point varies as a function of signal to noise, this is a property of the algorithm. A bias correction factor is applied to correct the position detected edge point. The bias correction factor is applied as a function of signal to noise.

The preferred embodiment implements the correction factor as a table which applied corrections at increments of 1% for various ranges of signal to noise which provides a smooth transition between corrections.

The signal power is estimated by taking the average of the FFT bin powers below the initial raw trace point over a short span of bins. The noise power is estimated from the noise model, averaging over Nₛₚₐₙ. The noise is estimated at the initial edge point.

Valve and Non-Flow Artefact Removal and Temporal Marker Extraction (102, 103 of FIG. 27)

The trace algorithm is applied on a point by point basis to generate a raw trace. As mentioned previously the Doppler spectra is contaminated by a number of extraneous, non-flow, sources. Artefact removal removes the non-flow sources to produce a trace representing the underlying flow. In this context artefact means anything which could contribute to unwanted flow on the spectral display.

The nature of the underlying flow profile and artefacts depend on the acoustic window used to acquire the target. In general a different scheme is required to remove the artefactual flow for each target type. The Pulmonary Valve (PV) target shows valve spike on the leading and trailing edges. The Aortic Valve (AV) target shows a tail region. When using CW Doppler both targets show artefacts from other flows and moving tissue in the acoustic field.

The flow profile can be considered as two images superimposed on one another: A flow trace showing the wanted flow, unwanted flow, and valve-clicks (which are stronger). Example valve clicks 30 and 31 can be seen in FIG. 11(a) which illustrates a PV waveform. At valve open, the valves may extend past the flow as shown in FIG. 11(a). Alternatively, if the flow has high enough velocity the valves may lie underneath the flow, or on the side of the flow as in FIG. 11(c). The valve and flow components are superimposed which makes it difficult to precisely identify the underlying flow profile. To remove the artefacts, the flow profile is therefore modified in an approximate manner.

The profile can be modified in many ways for example, fitted curves, piecewise approximations and lines. In a practical sense the accuracy of the modified profile need only be in same order, or below, the errors in the system or measurement method. For this reason forcing the profile using lines provides acceptable accuracy.

One of the key aspects of modifying the flow profile is identifying the portions of the velocity profile where the flow starts and ends. The temporal locations of these features can then be used derive temporal cardiac parameters, namely Heart Rate and Ejection Time. The wanted flow profile is bounded by valve activity. When the valve opens blood flows through the valve and when the valve closes the flow ceases. In addition, the flow associated with valve activity corresponds to a high slope on the velocity flow profile. Valve activity and flow slope are therefore good identifiers for the start and end of flow.

In order to perform the artefact removal in real time only a short portion of the most recent velocity profile is available for analysis.

Timing Marker Definitions

The heart timing marks 34 and 35 are shown in FIG. 11.

Heart Rate

The heart-rate is defined from the period from the start of valve-open (34) to the next start of valve-open. This definition approximates the R to R wave definition used by ECG machines since start of valve-open is largely synchronous with the R-wave.

Ejection Time

Ejection time is the duration of flow. In order to be consistent with the timing points used for heart rate, ejection time is the period from the start of valve-open region (34) to the end of the valve-closure region (35).

Implemented Algorithms

AV Algorithm 1

The AV algorithm works from velocity and velocity slope thresholds. It does not actively remove valve spikes because they are usually not present on an AV trace. The velocity threshold used for the valve closure is critical. For AV, the valve closure has a long tail which can become very flat. The valve-close point usually does not have a distinguishing feature in the tail.

Valve-Open

An approximate marker for valve-open is found using a velocity threshold, a robust slope threshold, and a glitch removal detector.

The raw trace must first exceed a preset valve-open velocity threshold. Using a number of points either side of this position, the slope of the trace is computed in a robust manner. If this slope exceeds a preset velocity slope threshold it is likely the position is the leading slope of the valve-open edge. The feature could however be atrial flow or a glitch. To make the leading edge detection more robust the algorithm looks ahead a number of points and makes sure the velocity remains above the valve-open velocity threshold, otherwise it is considered as non-flow and rejects the current point as a valve-opening. The point found on the edge of the flow is then used as an approximate marker position.

Since the edge is noisy the algorithm chooses points on either side of the approximate marker so the slope is maximized. The maximum velocity point is used as the end point for the forced profile. The minimum velocity point is used with the maximum velocity point extrapolating the edge line down to the zero velocity. The time position of the extrapolation is a reliable estimate for the start of valve-open which is forced to zero velocity. Note that the AV method does not need to find the end of valve-open, it only needs an edge point to modify the profile.

Valve-Close

The valve-closure algorithm is identical to the valve-open algorithm except that the direction of the slope threshold is reversed, and it uses a different velocity threshold and velocity change threshold. In addition there is a refinement step which attempts to move the end of valve closure point to a higher point on the curve to prevent extrapolating with points in the flat region of the tail.

PV Algorithm 1

The PV case is characterized by valve spikes at the start and end of flow. The valve spikes may or may not protrude above the underlying flow profile.

The start of the flow occurs at the start of the valve-open spike. If the valve spike does not protrude the valve edge may or may not overlap the flow. For the non protruding case it is not possible to detect the end of valve-open from the flow profile. The valve power is used to recover the otherwise hidden temporal information.

End of flow occurs at the centre of the valve-close spike.

Approximate Markers

The first phase of marker identification is to find approximate marker positions which are imprecise but relatively reliable. The algorithm uses these approximate markers for initial search points.

The velocity trace is low-pass filtered to remove any fine-grain bumps and glitches. The low-pass filter effectively integrates the flow profile to produce a near sinusoidal waveform which has a dip at the valve-open point and peak at the valve-close point. For PV, the valve closure spike is of sufficient magnitude to modify the sinusoidal shape. There can be a single peak which may be skewed either to the edge of flow, or, to the falling edge of the spike. Alternative there can be a peak corresponding to each of these cases with a dip in between corresponding to the notch in the flow between the flow and valve-close peak.

A peak detector is then passed over the filtered waveform to extract the peaks and identify the approximate marker positions as seen in FIG. 13. There is a negative going peak 40 just before the rising edge and a positive going peak 41 just after the falling edge.

Valve Power

The signal level for valve activity is generally strong. The signal power can therefore be used to identify valve activity.

Valve activity occurs simultaneously with flow. The total signal power comprises of three components: strong valve component, weaker flow component, and a weaker still noise component. The idea is to estimate these three power measures based on the FFT bin levels. Very strong signals above valve power threshold are classed as valve power, very small signals below a noise threshold are considered noise and the rest is considered flow.

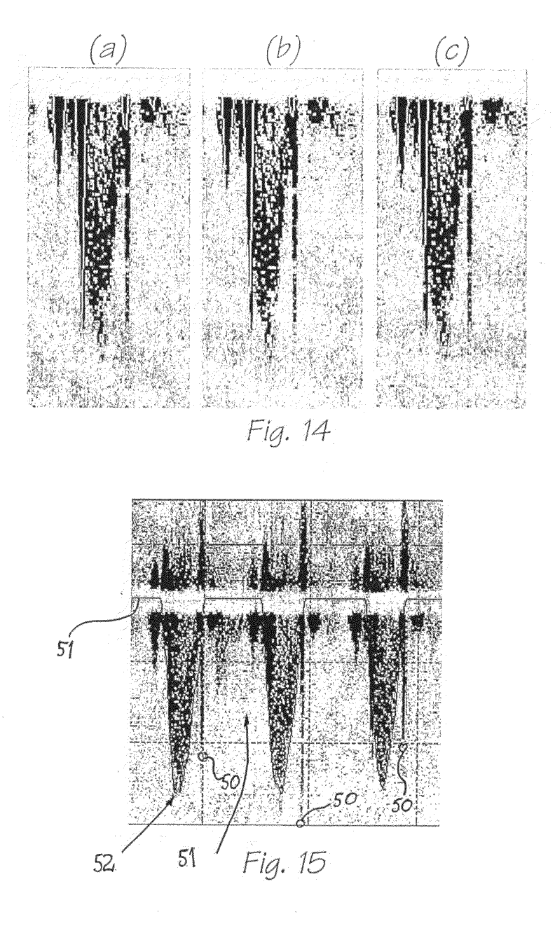

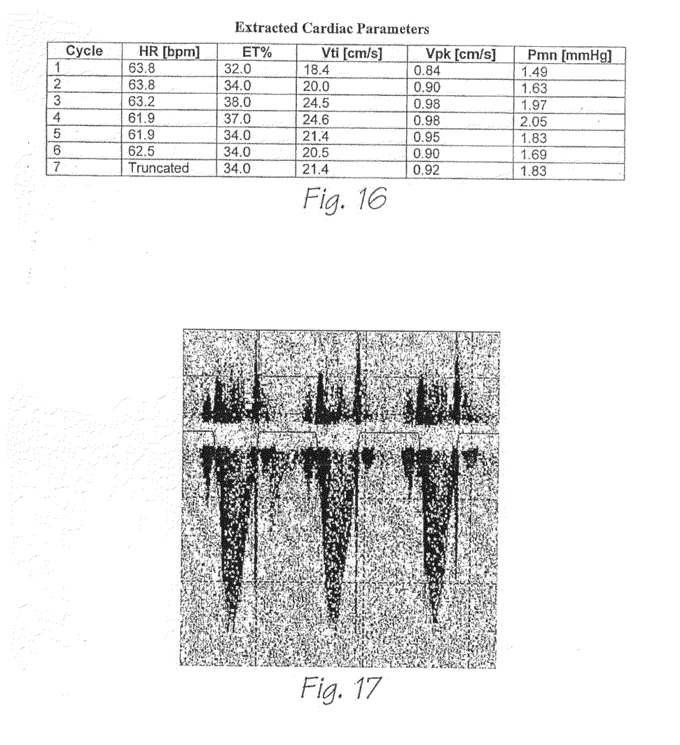

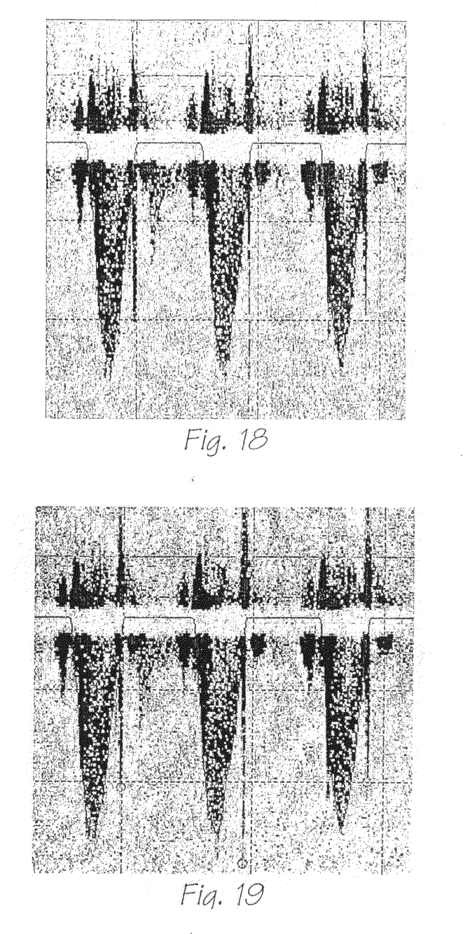

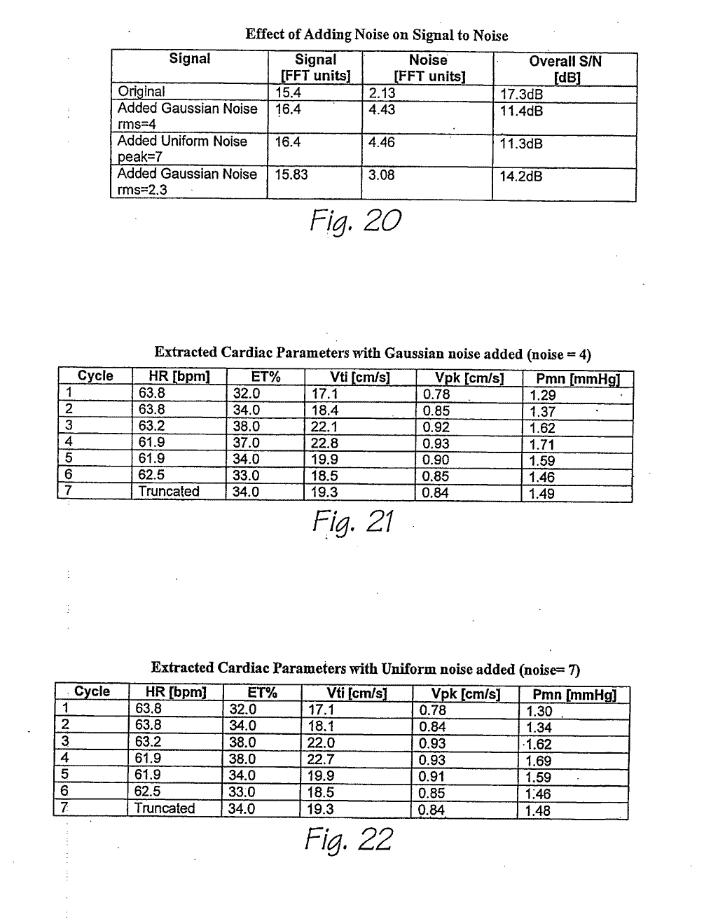

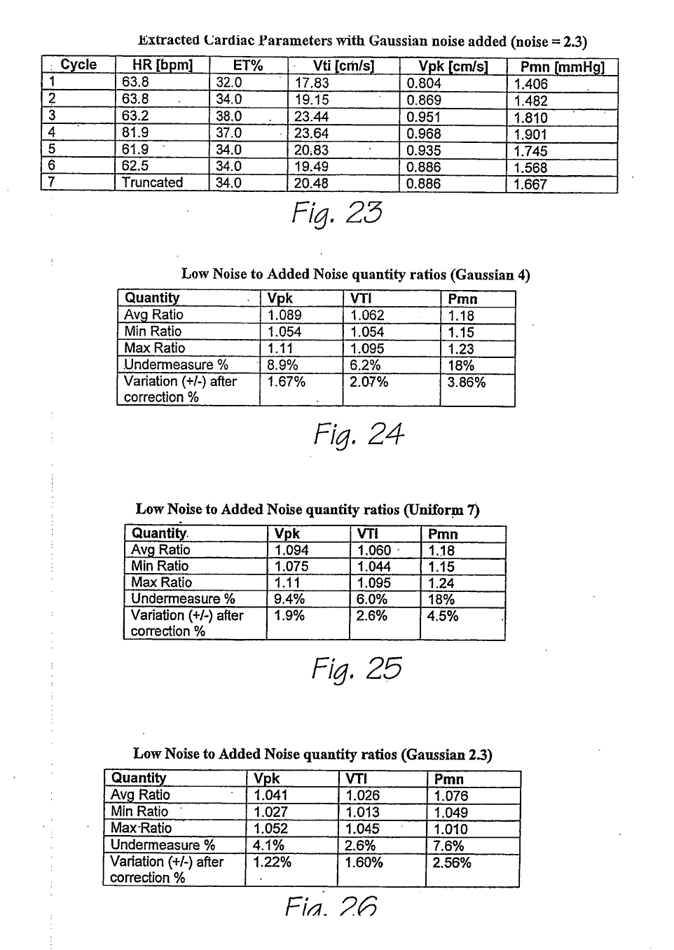

When total power includes all signal components, the power profile shows weak skirts at the base of the strong valve peaks. To remove the skirts a threshold must be applied to the power. An overall threshold or a bin threshold can be applied before computing the total power. The fixed bin-based threshold was chosen. FIG. 14 shows the improved indication of valve activity with the thresholded power and also the weakening of the lower power valves. Yet another alternative to identifying valve activity is to find the peaks in an unthresholded total power, with this method the total power threshold must be filtered using a short filter; say a three point FIR filter.

Ideally an adaptive threshold is required to discern between flow signal and valves. However, there will always be cases where the valve level is lower than the signal so an adaptive threshold will not solve all the problems.

The valve power is filtered by a two point moving average filter. This prevents single sample holes in the valve power affecting the zero power searches.

Wall Filter

The flow profile of the low velocity region contains many peaks and troughs. The large number of peaks is further exaggerated by the use of the raw trace. This region is known to contain irrelevant information. The algorithm removes these features to minimize the chance of finding false peaks. For analysis purposes, it is important to force the trace to the wall filter frequency and not zero flow. Forcing the flow to zero causes artificial high slope regions which cause false detections in the algorithm.

Valve-Open Removal

The flow profile is modified by forcing the velocity profile to a line to which is zero velocity at the start of valve-open to the point on the velocity flow profile at the end of valve-open. The valve-open period is identified as the region where the thresholded valve-power in non zero. The approximate valve-open marker position is used as a start point for the thresholded power search.

On occasion the valve start point is misjudged, usually because the valve-open power profile has leading small skirt. This can cause the forced valve-open profile line to be above the underlying trace. To minimise the error associated with misjudged points, the forced profile is prevented going above the underlying trace.

Valve-Closure Removal

The end of the valve-closure region is identified as the peak of the valve-closure spike in the velocity profile. The start of the valve-closure region is identified by the notch between the velocity flow profile and the valve-closure spike. The valve power for valve-closure is relatively weak and is not present in many cases, especially after thresholding the power, so a velocity spike was judged to be a more reliable indicator.

The algorithm uses the approximate valve-close end point to get a rough location of the valve closure region. It then identifies two peaks which are closest to approximate end marker. If the two peaks are too far from the approximate marker the approximate marker is used as the end point. If both peaks are close enough it uses the peak with the largest descent preceding the peak point over two samples. The raw trace can be very rough and there can be many spurious single sample peaks. The two-point span is more reliable and appears to work even if the valve closure is narrow. The largest preceding descent was found to be a good indicator of the valve closure peak in the velocity profile.

Once the end point is found, it uses the end point to find the notch between the valve-closure peaks and the main flow. For added robustness it was found to be better to allow the search go past the previously found end-point. If the start point found is after the end point the algorithm tries to find the valve activity based on valve power. If no significant valve power activity is found the end point is set to the estimate mark and the width of valve line is forced to 4 units.

Once the start and end points of the valve-close region are found, the flow is forced to a line which starts at the notch point and ends at zero flow at the end of the valve-close region.

The lines 32 and 33 on FIG. 11 show the underlying flow based on these definitions and linear approximation to the underlying flow profile; here these lines have been drawn manually. The chosen valve-closure points 50 are shown in FIG. 15.

PV Algorithm 2

This second algorithm follows from the ideas of the PV Algorithm 1 but uses a number of refinements.

One of the difficulties with PV Algorithm 1 is searching for the valves. There is uncertainty in the position of the approximate markers. The approximate markers may occur before or after the actual valve. Over a wide range of heart rates the trace filter distorts the positions of the approximate markers relative to the valves. These issues all require wide search spans which can introduce misdetection problems. Because this second method is more robust and relies less on searching unknown parts of the raw trace, it was found that the analysis wall-filter was no longer required. The second method does not rely on so much on the thresholded valve power to identify valve activity by using flow profile features where possible.

Valve-Open

The detection of the start of valve-open is the same as AV Algorithm 1 except different velocity and slope thresholds are used.

When using the thresholded valve power approach and a fixed power threshold, as per of PV Algorithm 1, the start of valve-open showed a large amount of variability across different people. With strong valve power the resulting width of the valve region caused early valve starts, in bad cases atrial movements were detected as the start of flow. A more reliable start of valve open was found by extrapolating the leading edge in the region around the approximate marker position down to the zero velocity point; as per AV Algorithm 1.

The end of valve-open is similar to PV Algorithm 1, it uses the approximate marker position as a start point to search for the end of the valve power region. The end of the valve power region is a first estimate for end of valve-open. A refinement step can be used to look ahead from this position on the velocity profile to see if the velocity is decreasing. The refinement is necessary because the valve-power end position is imprecise, and the position found could be on a valve spike, the refinement then tries to move down the valve spike.

Valve-Close

The valve close search uses some aspects of PV Algorithm 1 and improves others. The idea is to continuously monitor waveform features that occur after the valve-open edge. The waveform is largely processed in a left to right manner and decisions are made as the waveform is traversed. There are two main cases: where a valve spike is present, and where there is no valve spike. In the latter case a secondary search is done to make sure there is in fact no valve spike.

The first step makes sure the peak, and valve-open spike have been passed. Passing the peak is decided when the waveform height is below an end of peak threshold, which is still above the valve-close velocity threshold. When the region peak is passed the algorithm monitors the minimum velocity with the aim of finding the notch between the flow and the valve closure spike. This phase of the search terminates when a significant rise in the velocity profile is found, indicating the start of the valve-close spike. At that point the minimum velocity will be valid.

In parallel with this search is a second check to see if the velocity drops below a valve-close velocity threshold. If this check succeeds, either the valve is not visible (within the bounds of the defined thresholds), or, the notch point is at a very low velocity and the spike occurs past this point.

If the valve spike is detected the peak of the velocity profile for the valve spike is found. The peak position represents the end of valve-closed. Also, the notch position is refined by back searching for a lower velocity. The notch position becomes the start of valve-close. In this case the velocity profile is modified by forcing a line starting at the notch position down to zero velocity at the end of valve closure position.

If the valve spike was not detected, the trailing edge is extrapolated down using a method similar to the valve-open extrapolation. Starting from the higher-velocity point on the edge, a search is done for a sharp rising transition. The base of the transition is used as the notch point—this can be refined with a minimum search. From the notch point a search is done for the peak. If no transition is found the extrapolation points are used for the start and end of valve open; from the algorithm's point of view either there is no valve, or, it was smaller than the allowed thresholds and was missed.

Non-Flow Removal

The non-Flow region is simply the part of the Doppler spectrum 51 between the previous end of valve-closure and the current start of valve-open. Non-flow is removed by forcing the flow to be zero in this region.

Peak Velocity

The Peak Velocity 52 is the maximum velocity found between the start and end of flow of the current cardiac cycle. The peak is found after the valve-clicks are removed.

Algorithm Scaling

AV Algorithm 1 and PV algorithm 2 use a number of thresholds for feature detection. The thresholds can be adaptively scaled with heart rate. This is done using a “rough” heart rate detector which extracts the heart rate from the flow profile. Obviously other inputs, such as ECG, may be used to form the heart-rate input—such input could be used for approximate markers.

The rough heart rate detector can run completely independently of the trace algorithm. The motivation for using an independent detector is to prevent the algorithm feeding values into itself. Such feedback mechanisms can result in unstable behaviour or conditions where the algorithm locks itself out indefinitely. In addition, the independent information is a good way to cross check that signal conditions have stabilised.

The heart rate detector low-pass filters the raw velocity profile then uses a robust peak detector to detect the peaks in the filtered waveform. The time between the peaks is an estimate of the heart rate for that cycle. The most reliable peak is the negative going peak because this corresponds to the start of valve open. The valve open point has a well-defined edge and the valve spikes are less prominent. The robust peak detector incorporates a velocity threshold which excludes peaks which do not differ significantly from the previous

The heart rate detector averages a number of the beat to beat estimates to provide an average heart rate. The heart rate detector value is invalidated unless the following condition is met: A minimum number of heart beats have been acquired to ensure a reasonable amount of heart rate history. All heart rates used to form the average are within the allowed heart rate bounds, based on the average—this ensures all estimates are consistent with the check criteria.

Flow Trace Smoothing

Once the valve spikes in the flow profile are removed, the trace is smoothed using a 7-point FIR low-pass filter. A 7-point-60 dB Chebychev FIR filter was used for high rejection of the high frequency jagged edges.

The final smoothed trace 51 is shown in FIG. 14.

Unit Conversions and Formulas

It is assumed that the Doppler trace functions at a spoke rate of 10 ms. of the blood flow velocity through the heart, calculated from offsets values from the results of performing an FFT on the raw Doppler flow signal. The FFT offsets are referenced to a DC line (7 of FIG. 1) in the Doppler profile representing zero velocity. The following formulas are used in the trace function to extract the required cardiac parameters from the Doppler profile:

$$H R [ bpm ] = 60 / ( spokes between starts \times {t}_{spoke} )$$

$$E T \% = 100 \times spokes between start and end \text{/} spokes between starts$$

$$V T I [ cm \text{/} s ] = {k}_{fv} \times sum of trace heights between end and start$$

$${V}_{pk} [ cm \text{/} s ] = {k}_{fv} \times trace height of peak$$

$${P}_{mn} [ mmHg ] = 4 \times {k}_{fv}^{2} \times \frac{ \begin{matrix}sum of the square trace \\ heights between end and start\end{matrix}}{samples between start and end}$$

The factor k_{fv} depends on the Doppler frequency and the resolution of the frequency spectra, and, tₛₚₒₖₑ is the system dependent time between spokes.

The velocity profile is only available at discrete points. To perform the integrations for cardiac parameters VTI and Pₘₙ, and integral approximation must be made based on the finite points. The forced profiles ensure the velocity profile starts and ends at zero velocity. In this case the VTI and Pₘₙ summations are equivalent to a trapezoidal integration method. The numerical accuracy of the Trapezoidal method is deemed adequate. Other integration methods may be used.

FIG. 16 shows a typical set of extracted parameters.

1.-29. (canceled)

30. A method of determining the blood flow characteristics from a monitoring signal indicative of blood flow in a vicinity of a heart, the method comprising the steps of:
(a) extracting a flow envelope from the monitoring signal;
(b) removing extraneous flows such as valve opening and closing flows from the flow envelope;
(c) extracting a series of temporal markers from the flow envelope; and
(d) extracting features from the flow envelope and the monitoring signal.

31. The method of claim 30, wherein the features include a peak flow velocity and the monitoring signal comprises an ultrasound signal indicative of blood flow in the vicinity of the heart.

32. The method of claim 30, further comprising smoothing the flow envelope.

33. The method of claim 30, wherein the method provides for a real-time monitoring of blood flow characteristics of a patient's heart.

34. The method of claim 30, wherein the step of extracting a flow envelope from the monitoring signal further comprises extracting an expected maximum flow rate of fluid within the heart.

35. The method of claim 30, wherein the step of extracting a flow envelope from the monitoring signal further comprises:
(a1) forming a frequency domain transform of the monitoring signal; and
(a2) examining frequency domain characteristics of a power of the monitoring signal to make a determination of a maximum flow rate.

36. The method of claim 35, wherein the step of (a2) examining frequency domain characteristics of a power of the monitoring signal to make a determination of a maximum flow rate further comprises adjusting the determination in accordance with signal to noise levels in the monitoring signal.

37. The method of claim 30, wherein the step of extracting features from the flow envelope and the monitoring signal further comprises adjusting parameters in accordance with a perceived heart rate of the heart.

38. The method of claim 35, wherein the maximum flow rate is derived substantially from a maximum frequency signal level present.

39. The method of claim 30, wherein the step of (a) extracting a flow envelope from the monitoring signal further comprises utilising a slope threshold of the form:
mₜₕᵣₑₛₕ=kₛmₛ+k_{N}m_{N}
where kₛ is a signal weighting factor, k_{N} is a noise weighting factor, mₛ is a signal strength, and m_{N} is a noise level.

40. The method of claim 30, wherein the step of (a) extracting a flow envelope from the monitoring signal further comprises extracting the flow envelope from a power spectra of the monitoring signal and clamping a maximum value of the monitoring signal.

41. The method of claim 39, further comprising filtering a power spectra utilising a Kaiser filter.

42. The method of claim 39, further comprising filtering a power spectra utilising a modified Daniell filter.

43. The method of claim 39, further comprising filtering a power spectra in a frequency dependant manner.

44. The method of claim 39, further comprising filtering the power spectra in substantially an inverse frequency dependant manner.

45. The method of claim 30, further comprising examining the monitoring signal for artefacts and altering the monitoring signal to account for the artefacts.

46. A method of extracting cardiac parameters from a Doppler spectral display, the method comprising the steps of:
(a) detecting a Doppler spectral signal;
(b) extracting a flow envelope from the Doppler spectral signal
(c) removing undesired flow signals and artefacts from the flow envelope; and
(d) placing time markers at specific positions on the flow envelope to determine the cardiac parameters.

47. The method of claim 46, wherein the cardiac parameters are used to calculate further cardiac related parameters.

48. The method of claim 46, wherein the step of (a) detecting a Doppler spectral signal includes a Fast Fourier Transform (FFT) to produce a plurality of FFT magnitudes.

49. The method of claim 48, wherein the step of (b) extracting a flow envelope from the Doppler spectral signal further comprises:
(b1) determining a power spectra and an integrated power spectra from the FFT magnitudes;
(b2) estimating a noise power in the power spectra; and
(b3) determining a slope threshold in the power spectra corresponding to an actual flow represented by the Doppler spectral signal;
wherein bias correction factors scale an inaccurate quantity to a correct value without increasing sensitivity to noise.

50. The method of claim 46, wherein the cardiac parameters are extracted in real-time.

51. An automatic cardiac monitoring device comprising:
(a) a Doppler detection unit for detecting a Doppler spectral signal;
(b) a processing unit connected to the Doppler detection unit for processing the Doppler spectral signal to automatically extract a plurality of cardiac parameters, the processing unit identifying timing markers in the processed Doppler spectral signal to extract cardiac parameters; and
(c) a display unit interconnected to the processing unit for simultaneously displaying a visual representation of the processed Doppler spectral signal and the timing markers.

52. The device of claim 51, wherein the processing unit comprises:
a transfer function for extracting a flow envelope from the Doppler spectral signal; and
a transfer function for removing undesired flow signals and artefacts from a flow envelope.

53. The device of claim 51, wherein the cardiac parameters are extracted in real-time.

54. The device of claim 51, wherein the device is computationally compatible with fixed point DSP processors.
